(19) United States
(12) Patent Application Publication
(10) Pub. No.: US 2015/0157821 A1
(43) Pub. Date: Jun. 11, 2015

(54) COMBINED BITE BLOCK/ORAL AIRWAY/TONGUE DEPRESSOR APPLIANCE

(71) Applicant: THE REGENTS OF THE UNIVERSITY OF CDALIFORNIA, Oakland, CA (US)

(72) Inventors: Gerard R. Manecke, San Diego, CA (US); David J. Printz, San Diego, CA (US); An Vu, Oceanside, CA (US); William J. Mazzei, San Diego, CA (US)

(21) Appl. No.: 14/401,517

(22) PCT Filed: May 15, 2013
(86) PCT No.: PCT/US13/41185

§ 371 Date: Nov. 15, 2014

Related U.S. Application Data
(60) Provisional application No. 61/647,055, filed on May 15, 2012.
(60) Provisional application No. 61/784,097, filed on Mar. 14, 2013.

(51) Int. Cl. A61M 16/04; A61B 1/32; A61C 5/14; A61B 1/00; A61B 17/34; A61B 1/24; A61B 13/00

(52) U.S. Cl. CPC A61M 16/0495; A61B 1/24; A61B 1/32; A61B 13/00; A61M 16/0493; A61B 1/00154; A61B 17/3423; A61C 5/14; A61B 2017/345

(57) ABSTRACT

In particular, in alternative embodiments, the invention provides a combined bite block/airway/tongue depressor appliance. In alternative embodiments, appliances of the invention are used in an individual, e.g., an anesthetized patient or a patient having an electroconvulsive therapy (ECT), to maintain the airway. In alternative embodiments, appliances of the invention have a bite block and tongue depressor sufficiently asymmetrically positioned in the left- or the right-hand side of the mouth to allow additional positioning of a medical or visualizing device or an endotracheal tube into the mouth, the oral cavity, larynx or esophagus.

### TECHNICAL FIELD

This invention generally relates to anesthesiology. In particular, in alternative embodiments, the invention provides a combined bite block/airway/tongue depressor appliance. In alternative embodiments, appliances of the invention are used in an individual, e.g., an anesthetized patient or a patient having an electroconvulsive therapy (ECT), to maintain the airway. In alternative embodiments, appliances of the invention have a bite block and tongue depressor sufficiently asymmetrically positioned in the left- or the right-hand side of the mouth to allow additional positioning of a medical or visualizing device or an endotracheal tube into the mouth, the oral cavity, larynx or esophagus.

### BACKGROUND

Anesthetized patients are at risk of having their tongues fall back into their larynx, thus obstructing the airway. Currently used appliances often obstruct rather than open a patient's airway. Currently used appliances include are Guedel, Berman, and William airway products, and some are made from plastic, or foam products with different softness, and some are made with flexible plastic film with an air inflatable mechanism, and some are made with half plastic and half foam. Some airway are limited to assists air path for patient, some of the airways provide a bite block at the front teeth, some supplies with bite block only, some airway with tongue depressor airways. Some are not comfortable, and some hard to set up for use or took extra step to completed process.

### SUMMARY

In alternative embodiments, the invention provides combined bite block/oral airway/tongue depressor appliance, or a product of manufacture, comprising:

a bite block having sufficient width and length to maintain a spaced apart relationship between the upper and lower dentition;

a tongue depressor attached to or as part of the bite block (for example, a detachable piece), wherein the tongue depressor reaches or extends behind the tongue to prevent the tongue from blocking airflow during anesthesia, or the tongue depressor reaches or extends behind the tongue or sufficiently along the tongue to prevent the tongue from falling into a larynx and obstructing the airway; and

an air channel running or extending from the mouth opening through or along the full or partial length of the tongue depressor and reaching behind the tongue for airflow.

In alternative embodiments of the appliance or product of manufacture, the bite block is designed to have at least one pair of opposing molars or premolars bite onto the bite block to maintain a spaced apart relationship of the teeth, premolars or molars, and the bite block extends or is positioned on: (a) only on one side of the mouth and, wherein optionally the bite block extends or is positioned on only on the right side, or only the left side, of the mouth; or (b) both sides of the mouth.

In alternative embodiments, the bite block and tongue depressor are sufficiently asymmetrically positioned in the left- or the right-hand side of the mouth to allow additional positioning of an endotracheal tube into the mouth, or allow the introduction of a medical or visualizing device into the oral cavity, larynx or esophagus. In alternative embodiments, the bite block extends on both sides of the mouth substantially symmetrically and has at least one pair of opposing molars on each side of the mouth bite or contact onto the bite block to maintain a spaced apart relationship of the teeth or molars.

In alternative embodiments, the tongue depressor comprises a built-in airway running through substantially most of (the full length of) the tongue depressor, or through the center of the tongue depressor. In alternative embodiments, the tongue depressor comprises a built-in airway running through the center and full length of, or substantially most of (the full length of), the tongue depressor.

In alternative embodiments, the tongue depressor is removably attached to the bite block. In alternative embodiments, the distal end of the tongue depressor and/or the airway is sufficiently shaped and soft enough to prevent irritation, abrasion and/or damage to a soft palate when inserted into a mouth, wherein optionally the tongue depressor and/or the airway comprises or is made from a resilient polymeric material.

In alternative embodiments, the air channel airway extends outside of the mouth, and optionally has an adaptor for a gas, air or oxygen input, or the appliance or product of manufacture further comprises a tubular connector extending from the inlet port, or the air channel further comprises an oral outlet port.

In alternative embodiments, the appliance or product of manufacture of the invention further comprises a lip piece or a lip guard. In alternative embodiments, the bite block comprises material sufficiently soft to provide a cushion or a load distributing function when the teeth are biting or clenched onto the bite block; and optionally the appliance or product of manufacture, and/or the bite block, comprises or is made of a material or composition comprising a plastic, a Class VI Plastic, a foam, a biodegradable polymer, a polycarbonate, a soft or hard Polycarbonate, a SOFT POLYJET 95 DUROMETER SHORE A™, or equivalents or any combination thereof.

In alternative embodiments, the appliance or product of manufacture of the invention further comprises one or more orifices passing through the bite block, further comprising one or more orifices that allow passage through the appliance of a medical or visualizing device or an endotracheal tube.

In alternative embodiments, the invention provides appliances or products of manufacture as illustrated in the figures, including the devices or products of manufacture as illustrated in any of FIG. 1 to FIG. 12 (FIG. 1, FIG. 2, FIG. 3, FIG. 4, FIG. 5, FIG. 6, FIG. 7, FIG. 8, FIG. 9, FIG. 10, FIG. 11 or FIG. 12).

In alternative embodiments, the invention provides a combined bite block/oral airway/tongue depressor appliance or products of manufacture as illustrated in the figures, including the devices or products of manufacture as illustrated in any of FIG. 1 to FIG. 12 (FIG. 1, FIG. 2, FIG. 3, FIG. 4, FIG. 5, FIG. 6, FIG. 7, FIG. 8, FIG. 9, FIG. 10, FIG. 11 or FIG. 12).

In alternative embodiments, the invention provides methods of intubating a patient or a method for oral insertion of a medical devices or a visualization device, comprising use of an appliance or product of manufacture of the invention.

In alternative embodiments, the invention provides uses of an appliance or product of manufacture of the invention for intubating a patient or a method for oral insertion of a medical devices or a visualization device in a patient.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

Figures are described and discussed herein.

FIGS. 1 to 12 illustrate: the shape, design, form and construction of an exemplary product of manufacture of the invention, a Combined Bite Block/Airway/Tongue Depressor.

FIG. 1 illustrates an overall perspective view of an exemplary product of manufacture of the invention, a Combined Bite Block/Airway/Tongue Depressor.

FIG. 2 illustrates a top view perspective of a lower half an exemplary product of manufacture of the invention.

FIGS. 3 A, B, C, D and E schematically illustrate design features of an exemplary product of manufacture of the invention, with: FIG. 3A a top view perspective; FIG. 3B a front view perspective; FIG. 3C a bottom view perspective; FIG. 3D a midsection cutaway view perspective; and FIG. 3E a side view perspective. FIG. 3F is an overall perspective view of an exemplary product of manufacture of the invention, as schematically illustrated in FIG. 3A to 3E.

FIGS. 4 A, B, C, D and E schematically illustrate design features of an exemplary product of manufacture of the invention, with: FIG. 4A a top view perspective; FIG. 4B a front view perspective; FIG. 4C a bottom view perspective; FIG. 4D a midsection cutaway view perspective; and FIG. 4E a side view perspective. FIG. 4F is an overall perspective view of an exemplary product of manufacture of the invention, as schematically illustrated in FIG. 3A to 3E.

FIG. 5A illustrates an oblique side view perspective, and FIG. 5B illustrates a top view perspective, of an exemplary product of manufacture of the invention.

FIGS. 6 A, B, C and D schematically illustrate design features of an exemplary product of manufacture of the invention, with: FIG. 6A a top view perspective; FIG. 6B a front view perspective; FIG. 6C a bottom view perspective; FIG. 6D a side view perspective. FIG. 6E is an overall perspective view of an exemplary product of manufacture of the invention, as schematically illustrated in FIG. 6A to 6D.

FIG. 7 illustrates a top view perspective of an exemplary product of manufacture of the invention.

FIGS. 8A, B and C schematically illustrate alternative top view perspectives of an exemplary product of manufacture of the invention, showing exemplary dimensions.

FIG. 9 illustrates a cutaway view perspective of a side view of an exemplary product of manufacture of the invention.

FIG. 10 illustrates a side view perspective of an exemplary product of manufacture of the invention.

FIG. 11A to H schematically illustrate alternative perspectives of an exemplary product of manufacture of the invention: FIG. 11A illustrates a sectional side view perspective; FIG. 11B illustrates an oblique side view perspective; FIG. 11C illustrates an alternative top view perspective; FIG. 11D front view perspective; FIG. 11E illustrates a bottom view perspective; FIG. 11F illustrates an oblique side view perspective; FIG. 11G illustrates a sectional side view perspective; and FIG. 11H illustrates an oblique bottom view perspective.

FIG. 12A to H schematically illustrate alternative perspectives of an exemplary product of manufacture of the invention: FIG. 12A illustrates a top oblique side view perspective; FIG. 12B illustrates a cutaway sectional view perspective; FIG. 12C illustrates an alternative top view perspective; FIG. 12D a sectional side view perspective; FIG. 12E illustrates a bottom view perspective; FIG. 12F illustrates an top oblique side view perspective; FIG. 12G illustrates a front view perspective; and FIG. 12H illustrates an oblique bottom view perspective.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, compositions or products of manufacture of the invention comprise a combined bite block 102/tongue depressor 101 and airway 103 (Combined Bite Block/Airway/Tongue Depressor) 10 to maintain or open up an airway of an individual, e.g., an anesthetized patient or an individual undergoing electroconvulsive therapy (ECT), and prevent the tongue from falling into a larynx and obstructing the airway. In alternative embodiments, the appliance of the invention 10 comprises a tongue depressor 101 attached to or as part of the bite block 102, wherein the tongue depressor 101 reaches or extends behind the tongue to prevent the tongue from blocking airflow during anesthesia, or the tongue depressor 101 reaches or extends behind the tongue to prevent the tongue from falling into a larynx and obstructing the airway, e.g., during electroshock therapy or seizures.

In alternative embodiments, the bite block 102 of the appliance prevents the teeth from clenching down or contacting. In alternative embodiments, the bite block comprises a built-in airway 103 and tongue depressor (BBATD) 101. In alternative embodiments the bite block 102 is positions only on the, e.g., the molars, of one side of the mouth. In alternative embodiments, the appliance has an air channel 103 running through or alongside the tongue depressor 101 reaching behind the tongue for airflow. In alternative embodiments the device 10 is asymmetric with bite block 102 only on one side of it, thus providing enough space to incorporate an extended tongue depressor/airflow channel, and optionally, also having sufficient space for intubation or insertion of other medical devices or visualization devices.

In alternative embodiments, the bite block 102 is placed on the right or the left side of the month. In alternative embodiments, the appliance 10 is fitted for the size of the individual, e.g., a human patient, e.g., can have at least three or more different sized appliances for an adult population, and five, six or seven or more sizes for a youngster population, e.g., for humans, down to age 3. Each size can be designed to fit the anatomy of that particular age/size group. For example, for smaller sizes, the higher is the relative proportion of tongue/mouth cavity, thus the tongue depressor 101 is relatively longer. In alternative embodiments, the combination appliance product of the invention has a unique configuration and features in the design in order to function for individual patients, e.g., alternative (larger or smaller, wider or narrower) sizes of bite blocks 102, airways 103 (e.g., narrower) and/or tongue depressors 102.

In alternative embodiments, the appliance 10 comprises an outer lip protective ring 104. In alternative embodiments, the appliance 10 comprises an inner lip protective ring 105.

In alternative embodiments, the BBATD design for patients undergoing electroconvulsive therapy (ECT) may have a different configuration. Since there may be no need to allow space for intubation and the clenching force during the ECT may be stronger, in alternative embodiments the bite block 102 is on both sides of the mouth, e.g., symmetrically, and optionally the tongue depressor 101 has a built-in airway 103 running through the center of the device 10, e.g., the full length of the tongue depressor 101.

In alternative embodiments, the appliances 10 of the invention allow the bite block 102 to be easily inserted into the mouth while at the same time placing a bite block 102 between the teeth, e.g., the molars. In alternative embodiments, the shape of the invention's airway 103/bite block 102 allow it to be used when an endotracheal tube or other device is in place.

In alternative embodiments, the devices, appliances and products of manufacture 10 of the invention have a distal end soft enough to prevent damage to the soft tissue, e.g., the soft palate.

The invention provides devices, appliances and products of manufacture 10 that can be used as an airways product for use e.g., in hospitals or other medical care facilities, and also in an emergency care armamentarium, e.g., to assist and to keep air path open, e.g., to assist and to keep air path open during patient's intubation. In alternative embodiments, devices, appliances and products of manufacture 10 of the invention address shortcomings of existing appliances with one device to give every patient comfort and be effortless for healthcare personnel to use and to keep the patient's air way open and protect the patient's teeth, especially in psychiatric Electroconvulsive Therapy procedure.

In alternative embodiments, devices, appliances and products of manufacture of the invention are a Combined Bite Block/Airway/Tongue Depressor 10 for use with current practices to protect a patient's teeth in all cases including e.g., Electroconvulsive Therapy (ECT) procedures. In alternative embodiments, devices, appliances and products of manufacture 10 of the invention can assist medical personnel to intubate a patient. In alternative embodiments, devices, appliances and products of manufacture 10 of the invention are easy to set up and can minimize the step needed to achieve same goal compared to current use of multiple products.

In alternative embodiments, devices, appliances and products of manufacture of the invention, or any components thereof, are reusable or disposable devices.

In alternative embodiments, devices, appliances and products of manufacture of the invention, or any components thereof, can be manufactured by any known method, e.g., by injection molding, e.g., of a biologically inert, medical grade, latex free polymer or polymers. In alternative embodiments, devices, appliances and products of manufacture of the invention, or any components thereof, are made using biodegradable polymers, polycarbonates, soft or hard Polycarbonates, or equivalents, e.g., a SOFT POLYJET 95 DUROMETER SHORE A™.

In alternative embodiments, devices, appliances and products of manufacture of the invention, or any components thereof, are scaled to different proportional dimensions to accommodate the differing airway sizes of patients of all ages and physical dimensions. In alternative embodiments, devices, appliances and products of manufacture of the invention, or any components thereof, can be designed to have optimum dimensional relationships, to include variations in size, materials, shape, form, function and manner of operation, assembly and use, as are deemed readily apparent to one skilled in the art, and all equivalent relationships to those illustrated in the drawings and described in the specification are intended to be encompassed by the present inventive subject matter.

The following examples, and the figures, are intended to clarify the invention, and to demonstrate and further illustrate certain preferred embodiments and aspects without restricting the subject of the invention to the examples and figures.

### EXAMPLES

### Example 1

### An Exemplary Product of Manufacture: A Combined Bite Block/Airway/Tongue Depressor

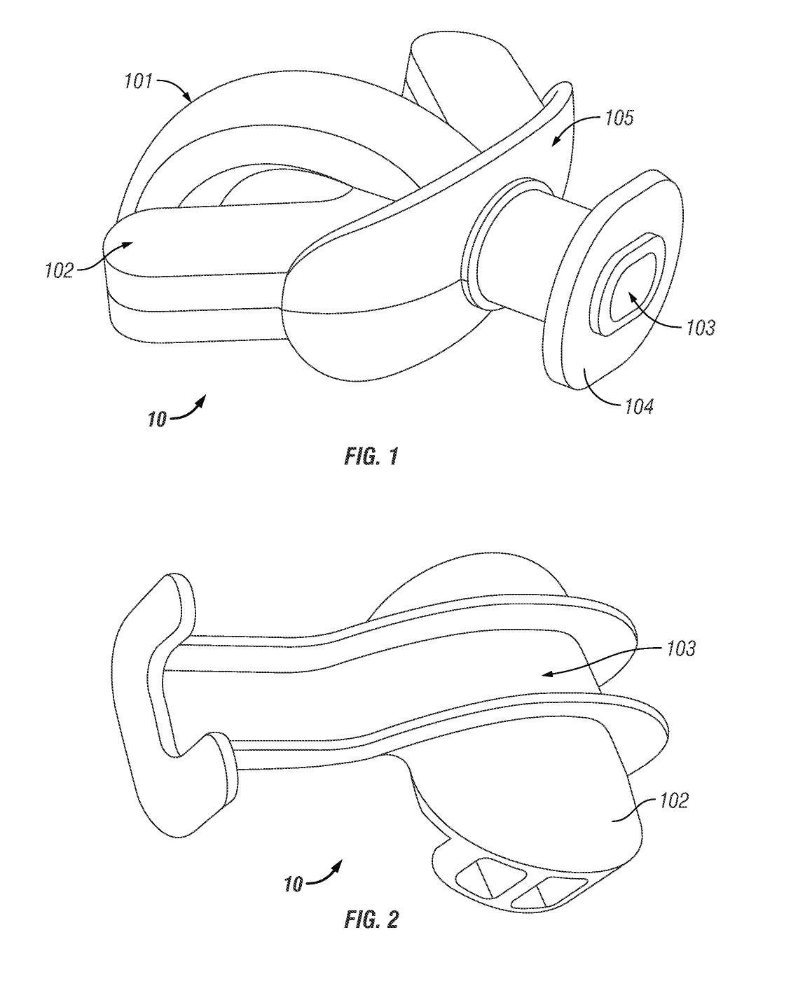

The following example describes, and FIGS. 1 to 12, illustrate, the shape, design, form composition and construction of alternative exemplary products of manufacture of the invention, embodiments of the Combined Bite Block/Airway/Tongue Depressor 10 of the invention:

### I. Design Embodiments, and Manufacturing Specifications

A. In alternative embodiments Combined Bite Block/Airway/Tongue

Depressor 10 designs are of several sizes, for example: 75 mm, 85 mm, 95 mm, to fit from small children to large adult patient. In alternative embodiments, the size of the airway can be between about 70 to 100 mm, or between about 75 to 90 mm, or between about 80 to 85 mm.
B. In alternative embodiments a Combined Bite Block/Airway/Tongue Depressor 10 will be made from different materials and with softness. In alternative embodiments, two materials that can be used are (or their equivalents):
   a) Hard Polycarbonate.
   b) Soft PolyJet 95 Durometer Shore A
C. In alternative embodiments a Combined Bite Block/Airway/Tongue Depressor 10 design gives patient comfort with an ease of set up for the operating room personnel.
D. In alternative embodiments Combined Bite Block/Airway/Tongue Depressor 10 is designed or manufactured with a medical grade material.
E. In alternative embodiments Combined Bite Block/Airway/Tongue Depressor 10 shall be applied with FDA 510(K) and registered.
F. Combined Bite Block/Airway/Tongue Depressor 10
G. In alternative embodiments Combined Bite Block/Airway/Tongue Depressor 10 is designed with different colors to differentiate sizes of the product, the airway, and/or the bite block and/or the like.
H. In alternative embodiments the Combined Bite Block/Airway/Tongue Depressor 10 is manufactured/designed and/or packaged to be non-sterile, or sterile.

### II. Reliability Embodiments

A. In alternative embodiments a Combined Bite Block/Airway/Tongue Depressor 10:
   1. is designed for medical personnel to monitor the patient's airway path and to prevent the patient's teeth damage during and intubation. In alternative embodiments it is Non sterile, or sterile, and/or for single use only. In alternative embodiments it is made of a material that is sterilizable and/or autoclavable for reuse.
   2. In alternative embodiments is designed of several sizes to fit from child to large adult.

### III. Interface Embodiments

A. In alternative embodiments a Combined Bite Block/Airway/Tongue Depressor 10 is design with no interface devices, or having one or multiple interface devices.

### IV. Manufacturing Embodiments

A. In alternative embodiments a Combined Bite Block/Airway/Tongue Depressor 10 design gives patient comfort with an ease of set up for the operating room personnel.
B. In alternative embodiments the Combined Bite Block/Oral/Tongue Depressor/Airway is sterile, or cleaned.
C. In alternative embodiments the Combined Bite Block/Airway/Tongue Depressor 10 is coded for size, for example, is color coded.
D. In alternative embodiments a Class VI Plastic is used and a biocompatibility test for a Class VI Plastic is completed.
E. In alternative embodiments a Combined Bite Block/Airway/Tongue Depressor 10 is designed as one piece, or a unibody, or is designed as multiple pieces, which alternatively can be disassembled for cleaning, replacement or customizing for a particular patient, a particular need, or for a customized or a particular attachment, e.g., an endoscopic tube.

A number of embodiments of the invention have been described. Nevertheless, it will be understood that various modifications may be made without departing from the spirit and scope of the invention. Accordingly, other embodiments are within the scope of the following claims.

1. A combined bite block/oral airway/tongue depressor appliance, or a product of manufacture, comprising:
a bite block having sufficient width and length to maintain a spaced apart relationship between the upper and lower dentition;
a tongue depressor attached to or as part of the bite block, wherein the tongue depressor reaches or extends behind the tongue to prevent the tongue from blocking airflow during anesthesia, or the tongue depressor reaches or extends behind the tongue or sufficiently along the tongue to prevent the tongue from falling into a larynx and obstructing the airway; and
an air channel running or extending from the mouth opening through or along the full or partial length of the tongue depressor and reaching behind the tongue for airflow.

2. The appliance or product of manufacture of claim 1, wherein the bite block is designed to have at least one pair of opposing molars or premolars bite onto the bite block to maintain a spaced apart relationship of the teeth, premolars or molars, and the bite block extends or is positioned on:
(a) only on one side of the mouth and, wherein optionally the bite block extends or is positioned on only on the right side, or only the left side, of the mouth; or
(b) both sides of the mouth.

3. The appliance or product of manufacture of claim 2, wherein the bite block and tongue depressor are sufficiently asymmetrically positioned in the left- or the right-hand side of the mouth to allow additional positioning of an endotracheal tube or equivalent into the mouth, or allow the introduction of a medical or visualizing device or equivalent into the oral cavity, larynx or esophagus.

4. The appliance or product of manufacture of claim 2, wherein the bite block extends on both sides of the mouth substantially symmetrically and has at least one pair of opposing molars on each side of the mouth bite or contact onto the bite block to maintain a spaced apart relationship of the teeth or molars.

5. The appliance or product of manufacture of claim 1, wherein the tongue depressor comprises a built-in airway running through substantially most of the tongue depressor, or through the center of the tongue depressor.

6. The appliance or product of manufacture of claim 4, wherein the tongue depressor comprises a built-in airway running through the center and full length of, or substantially most of, the tongue depressor.

7. The appliance or product of manufacture of claim 1, wherein the tongue depressor is removably attached to the bite block.

8. The appliance or product of manufacture of claim 1, wherein the distal end of the tongue depressor and/or the airway is sufficiently shaped and soft enough to prevent irritation, abrasion and/or damage to a soft palate when inserted into a mouth, wherein optionally the tongue depressor and/or the airway comprises or is made from a resilient polymeric material.

9. The appliance or product of manufacture of claim 1, wherein the air channel airway extends outside of the mouth, and optionally has an adaptor for a gas, air or oxygen input, or the appliance or product of manufacture further comprises a tubular connector extending from the inlet port, or the air channel further comprises an oral outlet port.

10. The appliance or product of manufacture of claim 1, further comprising a lip piece or a lip guard.

11. The appliance or product of manufacture of claim 1, wherein the bite block comprises one or more materials sufficiently soft to provide a cushion or a load distributing function when the teeth are biting or clenched onto the bite block.

12. The appliance or product of manufacture of claim 1, further comprising one or more orifices passing through the bite block, further comprising one or more orifices that allow passage through the appliance of a medical or visualizing device or an endotracheal tube.

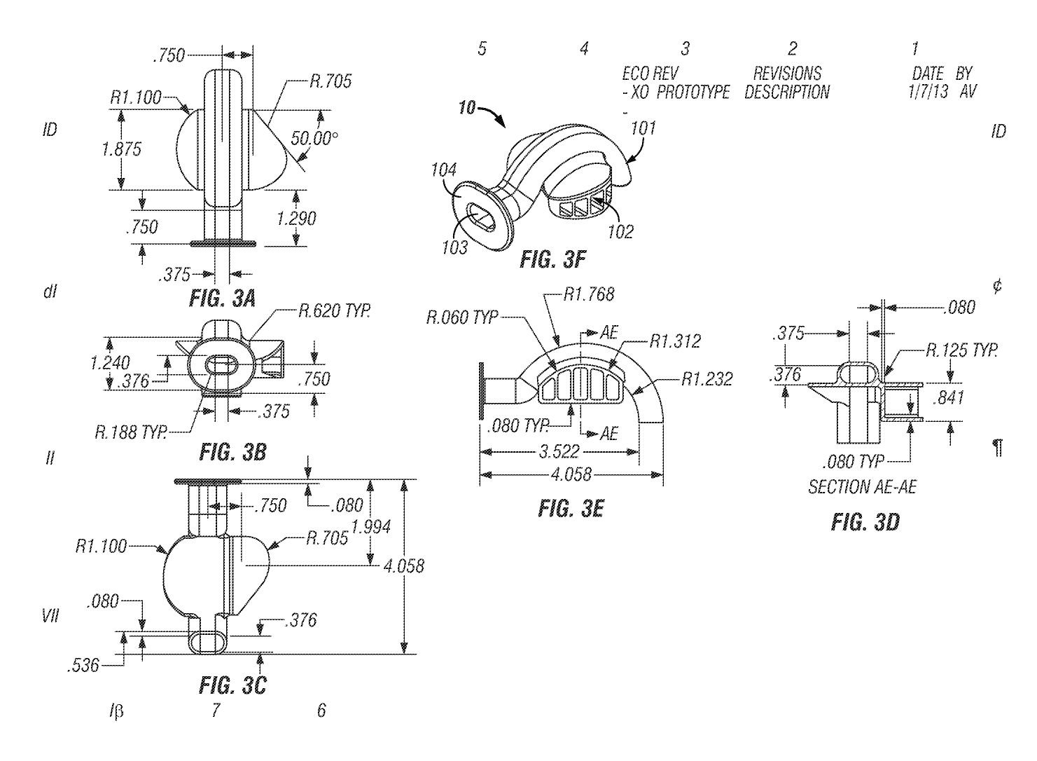

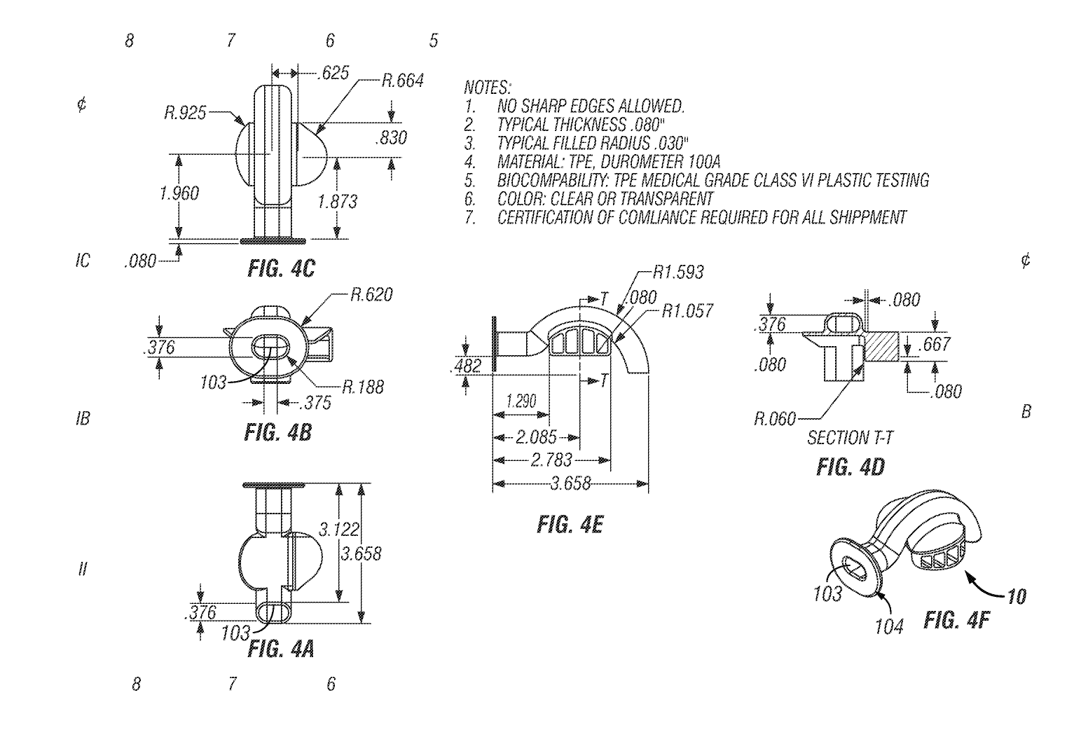

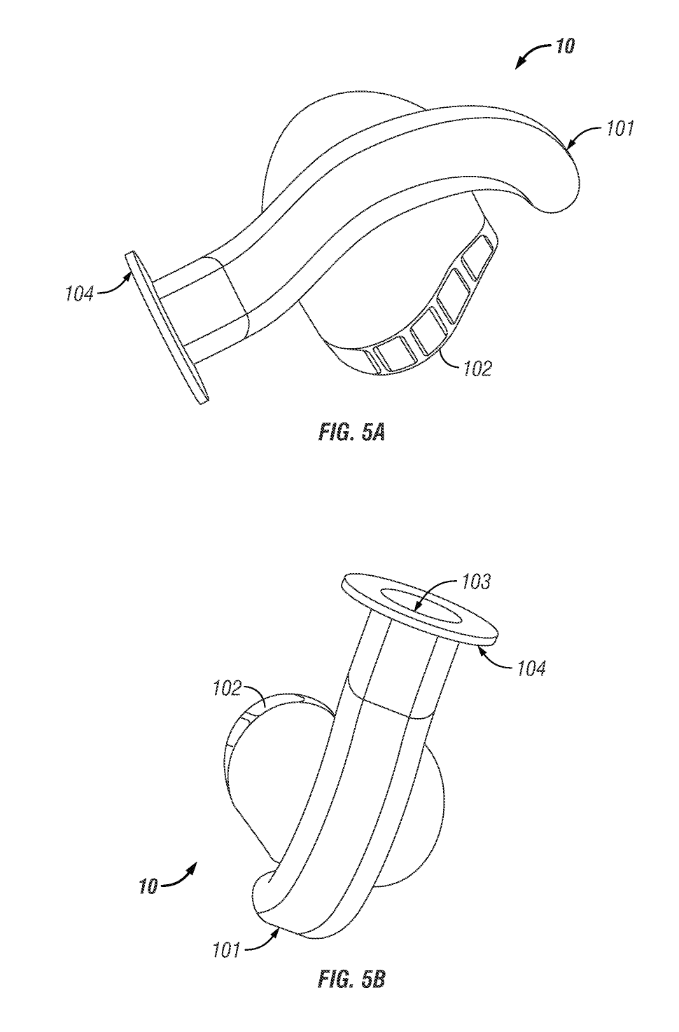

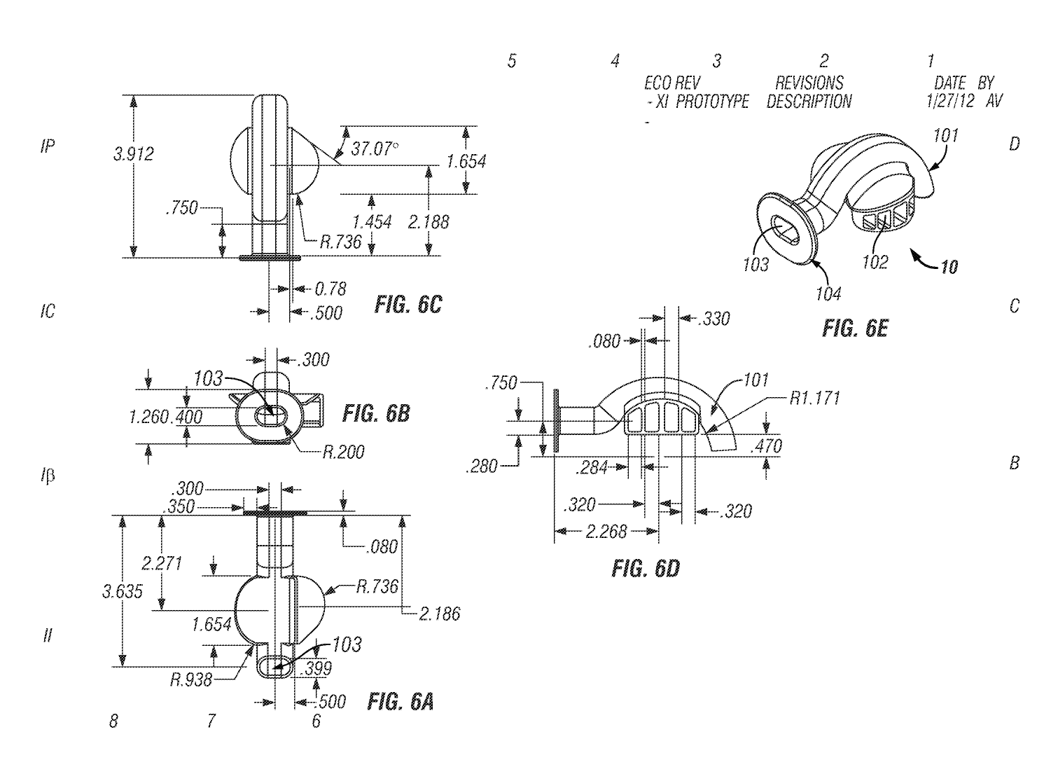

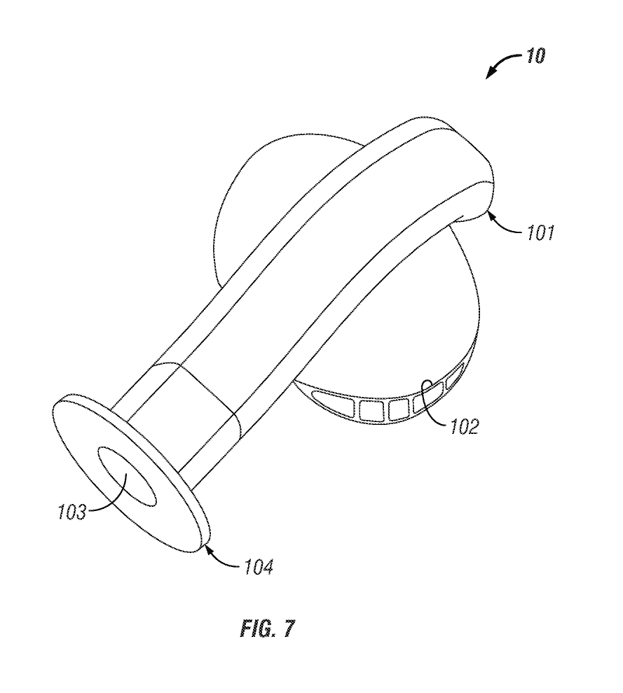

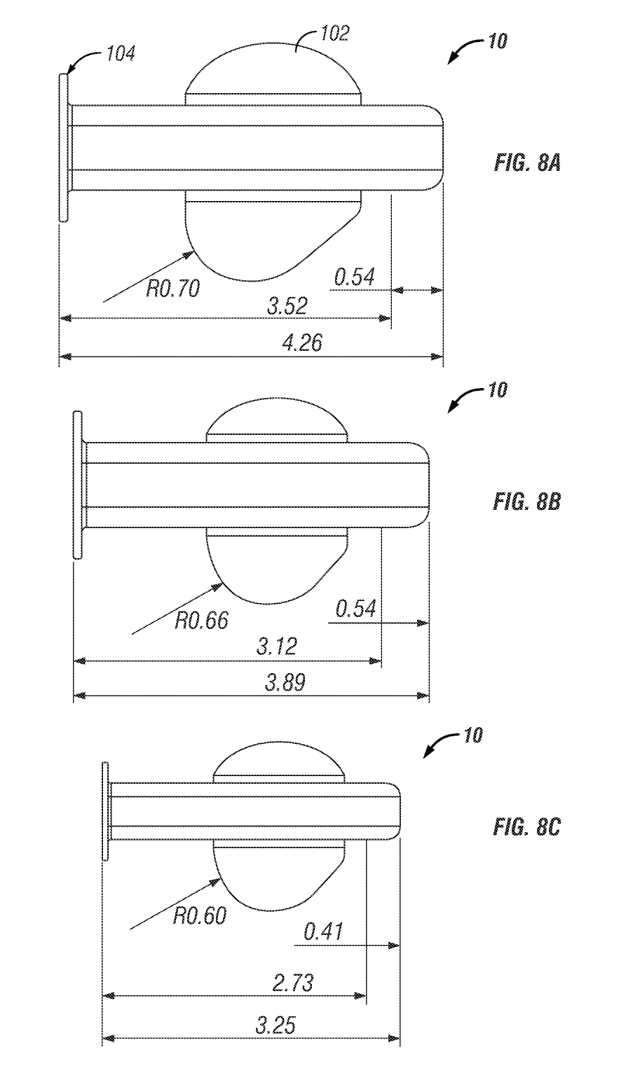

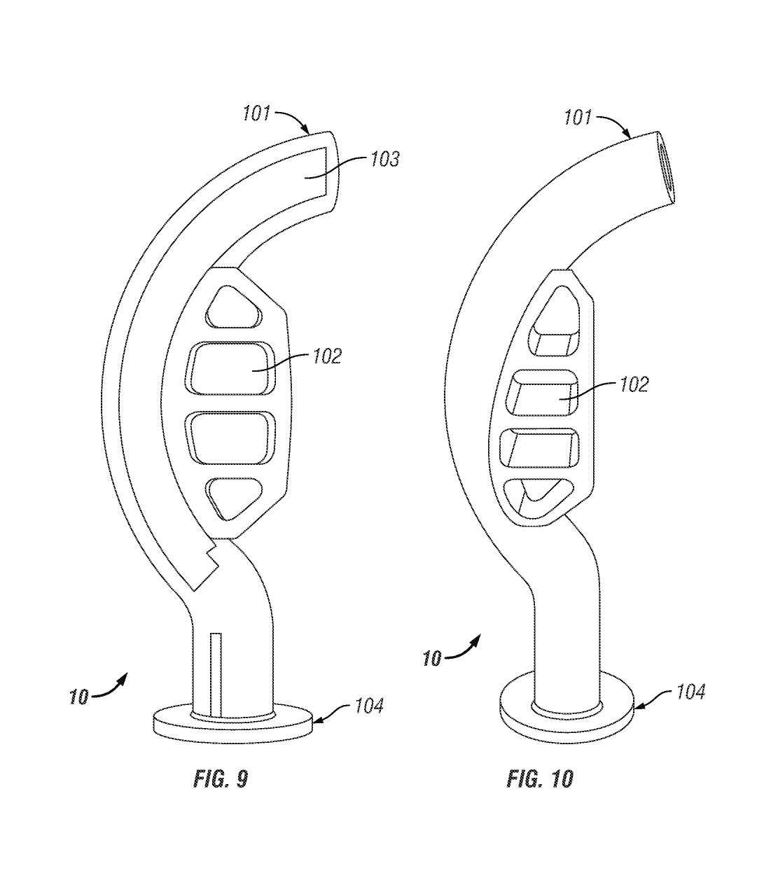

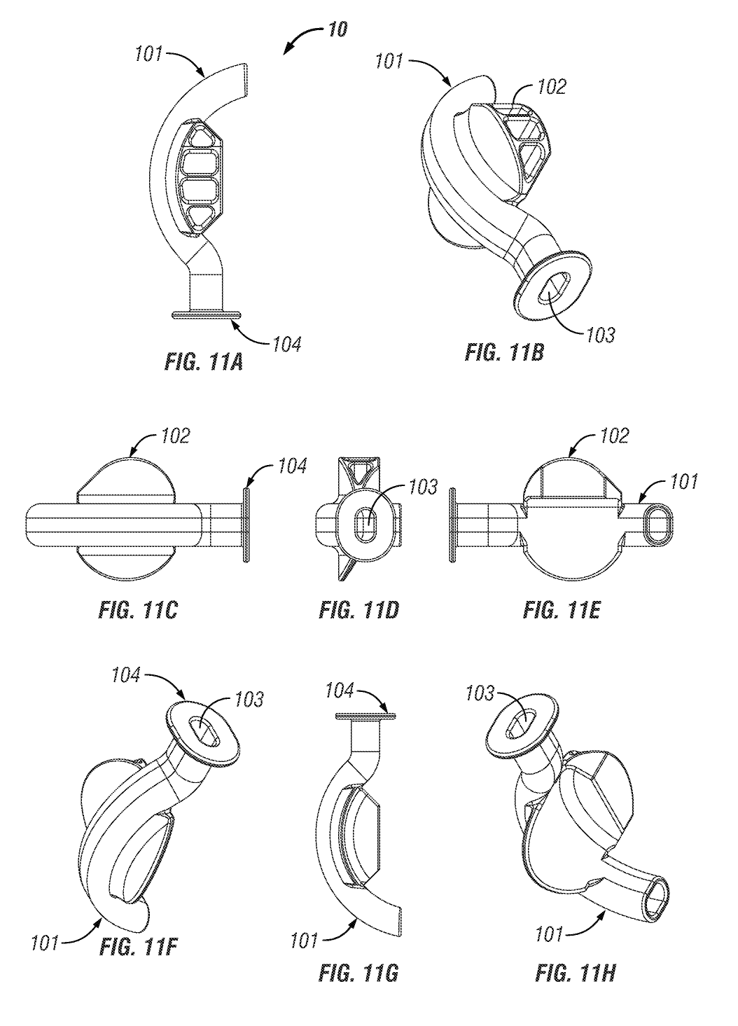

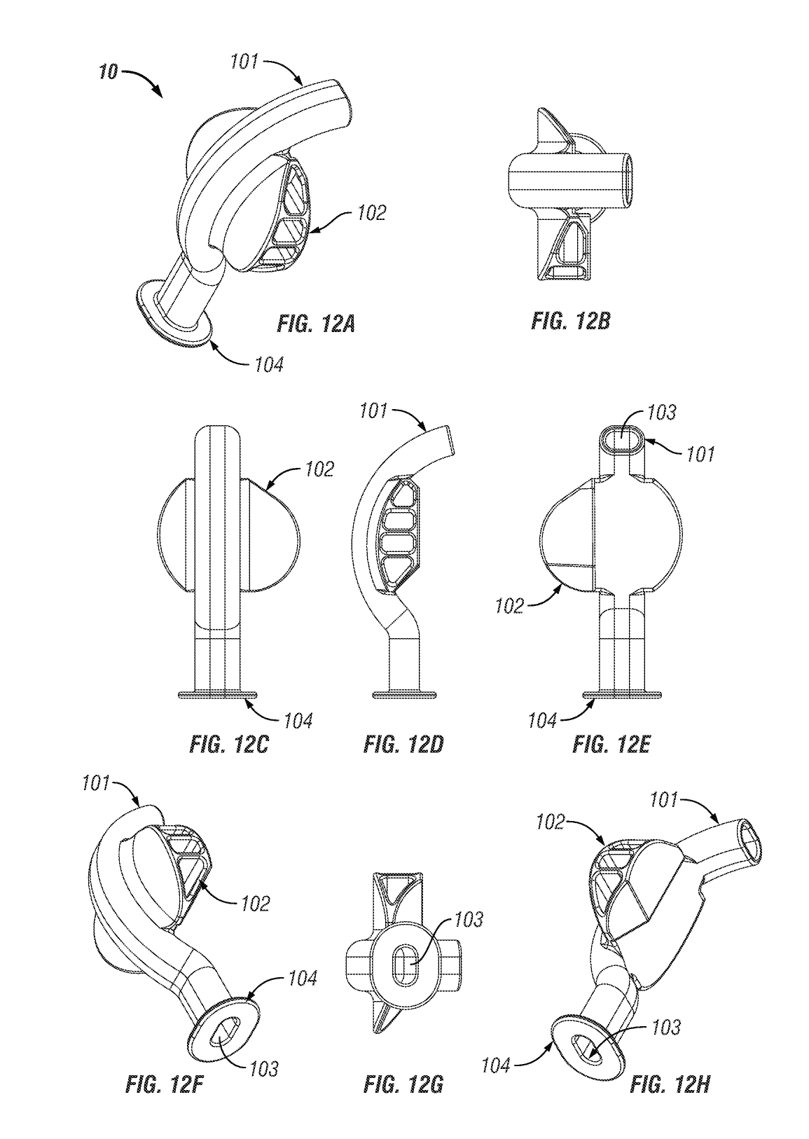

13. The appliance or product of manufacture of claim 1, wherein the appliance or product of manufacture is the device or product of manufacture illustrated in FIG. 1, FIG. 2, FIG. 3, FIG. 4, FIG. 5, FIG. 6, FIG. 7, FIG. 8, FIG. 9, FIG. 10, FIG. 11 or FIG. 12.

14. A combined bite block/oral airway/tongue depressor appliance or product of manufacture comprising a device or product of manufacture as illustrated in FIG. 1, FIG. 2, FIG. 3, FIG. 4, FIG. 5, FIG. 6, FIG. 7, FIG. 8, FIG. 9, FIG. 10, FIG. 11 or FIG. 12.

15. A method of intubating a patient or a method for oral insertion of a medical devices or a visualization device, comprising use of an appliance or product of manufacture of claim 1.

16. (canceled)

17. The appliance or product of manufacture of claim 1, wherein the appliance or product of manufacture, and/or the bite block, comprises or is made of a material or composition comprising a material selected from the group consisting of: a plastic, a Class VI Plastic, a foam, a biodegradable polymer, a polycarbonate, a soft or hard Polycarbonate, a SOFT POLYJET 95 DUROMETER SHORE A™, and a combination thereof.
